(19) United States
(12) Patent Application Publication
(10) Pub. No.: US 2021/0272676 A1
(43) Pub. Date: Sep. 2, 2021

(54) SURGICAL DEVICE AND CONTROL METHOD THEREFORE

(71) Applicant: ERBE ELEKTROMEDIZIN GMBH, Tuebingen (DE)

(72) Inventor: Christof Dierl, Bondorf (DE)

(73) Assignee: ERBE ELEKTROMEDIZIN GMBH, Tuebingen (DE)

(21) Appl. No.: 17/177,423

(22) Filed: Feb. 17, 2021

(30) Foreign Application Priority Data
Feb. 27, 2020 (EP) 20159760.6

(51) Int. Cl. G16H 20/40; G16H 40/63

(52) U.S. Cl. CPC G16H 20/40; G16H 40/63

(57) ABSTRACT

A device for surgical treatment of patients having an apparatus with an electrical generator at which different modes can be selected. In addition to selection of provided modes and to the adjustment of their effect strength, the authenticated treating person can generate at least one individual mode and assigned individual mode data for himself and, if applicable, also for selected colleagues as re-users in the apparatus, based on which the generator operates beyond the certified modes.

### CROSS REFERENCE TO RELATED APPLICATION

This application claims priority to European Application No. 20159760.6, filed Feb. 27, 2020, the entirety of which is incorporated by reference herein.

### TECHNICAL FIELD

Embodiments of the invention are a device for surgical treatment of animal or human patients as well as method for operating a treatment device.

### BACKGROUND

Instruments are used in electrosurgery that are supplied with current and voltage from a supplying apparatus in order to create a desired effect on the patient, e.g. a coagulation, a cut or the like. For this the surgical apparatus comprises a radio frequency generator for creating a treatment current and a typically program-controlled control device in order to operate the generator according to predefined modes.

For this DE 10 2009 042 428 B4 discloses a method for creation of a control program for such a surgical generator. The method is based on the illustration of states that have to be adapted by the surgical generator depending on predefined events in the form of a state machine. The state machine is then translated into a control program by means of which the instrument and/or the generator is/are controlled.

The state machine defines conditions that, if they occur, characterize the transition of a predefined state in another defined state of the system. The state machine, however, does not allow the transition to states that do not occur in itself. This is a limitation with regard to states that can be selected by the user. The same applies, if only predefined modes are provided for selection for the user by a user interface of an apparatus and in the context of one mode the effect strengths can be adjusted, as is usual in practice. Also here the user can finally only select between predefined states. The predefined states are approved and documented in their effects and thus legally safe. A direct access on physical values of the treatment current is thus not provided.

Further a method for operating an electrosurgical generator is known from EP 3 028 657 A1, with which algorithms for control of an electrosurgical generator can be provided. These algorithms use so-called configuration files that can be modified and processed by means of an interpreter. The control of the generator is thus based on the configuration files. Physical parameters, like for example power, current, voltage, energy, coagulation parameters or the like can be adjusted by a user interface, e.g. on a scale from 1 to 10 or 1 to 5. The configuration files can be provided in the apparatus or for example a cloud-based update server.

In addition, EP 3 028 657 A1, WO 2018/210792 A1, EP 1 337 194 A1 and EP 1 617 776 B1 show aspects of the control of surgical generators based on algorithms. Particularly the programming of an electrosurgical generator by a user by means of program commands is known from EP 1 617 776 B1. The so created control software can be stored on mobile computer readable media and/or can be received or transmitted by signal transmission.

### SUMMARY

It is the object of embodiments of the invention to provide a device for surgical treatment of animal or human patients as well as a method for control of such a device by which on one hand the necessary treatment safety and on the other hand a minimum limitation of the treating physician in terms of his medical measures can be achieved.

The device according to claim 1 and the method according to claim 9 fulfill these per se contrary requirements:

An embodiment of the inventive device comprises an apparatus for supply of at least one instrument. The apparatus comprises a generator that provides a voltage for the instrument as well as the electrical current serving the supply of the instrument. The generator is controlled by a control device that provides modes that can be selected by a treating person for selection by the treating person. Mode data are part of each mode that define a number of physical parameters of the current and/or the voltage. The control device is configured to control the generator according to the mode data of a mode selected by the treating person.

Further the apparatus comprises a selection device by means of which the treating person can manually select a mode. In addition, the selection device provides a possibility for adjustment of a desired effect strength for at least one, preferably multiple or all of the selectable modes.

In addition, the apparatus can be connected with a memory arranged outside the apparatus via a suitable data connection. The memory can be, for example, a cloud, a web server or another memory location that can be reached via a data connection. The data connection is particularly a data network, e.g. a private or public data network, the internet or the like. Individual mode data can be stored in the memory that are assigned to individual modes that are individually assigned to selected treating persons. For this identifiers are assigned to the individual mode data. The respective identifier indicates which rights a selected treating person has on these individual mode data. Rights can be, for example, the authority to modify individual mode data and/or to use individual mode data.

The apparatus comprises an authentication device by means of which the treating persons can be individually identified. The treating person can use the individual mode data according to his rights after authentication, i.e. for example, modify them and transfer them to an apparatus in order to use them there. Alternatively, a less privileged user that is only authorized for reuse can be authenticated to transfer the individual mode data for use on his apparatus.

In addition, the authentication device can be configured to register a use of individual mode data and if desired or necessary, transmit it to an accounting unit. The authentication is further configured to block or delete individual mode data after termination of the authentication.

With this device the previous safety standards are complied with and also additional treatment options are opened up. The usual modes are selectively provided for a non-authenticated user, however, and can adjust effect strengths for the selected modes. An authenticated treating person can, if he has the highest authentication level, create and/or modify individual mode data and has thereby direct access on physical parameters of the voltage and/or the current, as well as further parameters such as, for example, treatment duration, maximum power, maximum energy, feedback control parameters, evaluation of an instrument recognition, etc. The so created individual mode data are solely usable for this one treating person to whom the data “belong”. The treating person can use these individual mode data in the context of the medical freedom of treatment without specific certification for his activity. He can also create a data sheet, i.e. a description of the individual mode created by him or have the data sheet created and can link this description with the individual mode data. Thus, he can make the individual mode data created by him accessible for re-users that obtain the right after authentication to load the individual mode data on their apparatus and to use them there, but not to modify them. Together with the individual mode data they can download the data sheet, i.e. the mode description, and can then use this individual mode in the context of their medical freedom of treatment. The use can be detected and can be debited by an accounting module by way of a licensing model or other accounting models. The reuse of individual mode data can be registered and can be made available to the owner of the individual mode data.

It is in general possible to detect the use of specific modes, the duration of the use, the number of uses or the like of standard modes or also of individual modes and to generate respective data therefrom. It can be provided that these data are transmitted to a central server via data connection. These data can be the basis for an accounting model that charges the user for the use of an apparatus or instrument. If the data are anonymized, they can serve for statistical determination of the use or of modes.

In order to avoid a non-authorized reuse of individual mode data and the dangers for personnel and patient accompanied therewith, the authentication device preferably comprises a check-out option that inhibits use of individual mode data after check-out of the authenticated treating person. The check-out option can be, for example, an option provided on a screen. Alternatively, it can be connected with the on/off switch of the apparatus such that after switching off the apparatus, individual mode data are only accessible after authentication of authorized treating persons. This is independent from whether the individual mode data are memory-resident maintained in the apparatus or whether they have to be downloaded again from a memory provided external from the apparatus.

The assignment of individual mode data to different treating persons and the administration of rights thereof allows that authenticated treating persons can have access to their individual mode data at any location, e.g. via the internet, independent from the physical apparatus with which they work, independent from the operating room in the hospital or any other local circumstance. The user can download data on their apparatus configured for this in order to be able to use the treatment options developed by them.

For support of generation of individual modes, a mode generator can be provided that detects user inputs of an authenticated treating person with respect to physical parameters of the voltage and/or the current or other values (power, work, crest factor, etc.) and generates individual mode data therefrom. The mode generator allows access to multiple physical parameters of the provided voltage or current with or without professional advice of trained experts in a supporting central department, at which preferably also the memory for storing the individual modes is located. Physical parameters of the voltage and/or the current can be, for example:

The peak voltage, the duty cycle of the voltage and/or the current, the switch-on duration, the applied electrical work, the maximum power, a parameter characterizing the strength or quality of a created spark, the minimum power, an envelope shape of the voltage or the current, the modulation forms, the modulation depth, the time-dependent variation of one of the above-mentioned parameters, the variation of one of the above-mentioned parameters depending on measurement values, such as, for example, current, voltage, resistance, etc.

The user has access to such physical parameters of the current or the voltage by means of the mode generator that otherwise are excluded from access of the treating person. In doing so, the possibility is provided that treating persons (i.e. physicians or also other users) develop modes from their medical experience and provide them for use by themselves as well as publish such modes and make them available for other physicians (re-users).

Individual modes can be created, for example, via a program or an app of an external computer. It can be carried out on a server, on an external computer connected with the server, an apparatus connected with the server or this computer by an external expert or at the surgical apparatus itself by the treating person or an assisting person. The external computer can then send the individual mode data directly to the server or can transfer them first to the surgical apparatus that then transmits the data to the server.

For example, individual modes can be based on predefined modes. For example, an apparatus of the applicant provides a mode “preciSECT” that serves for operation of anatomic forceps. This mode is characterized in that the maximum voltage and the crest factor is controlled by the resistance value measured at the forceps. This mode cannot be well applied in laparoscopy, because it leads to undesired results in case of unintentional contact of metal trocars. Having the possibility to create individual mode data, a treating person can create his own individual mode that avoids disturbing effects during laparoscopic application.

Also some users desire a greater hemostasis and/or less cutting characteristics of the instrument, e.g. an electro-scalpel. The user can achieve this by adaption of crest factor and power limitation that are usually not accessible as adjustment parameters.

Another example is the mode “EndoCutQ” provided by the applicant. This mode provides a voltage increase over three cutting pulses in order to not cut too quickly during polyp removal with loops. If he intends to use this mode during endoscopic submucosal dissection (ESD) with a needle instrument, this is disturbing for some users, because now it shall not be cut initially slightly delayed. By generating of an individual mode on the basis of “EndoCutQ” the treating person himself can provide remedy quickly.

Another example is the mode “SwiftCOAG” provided by the applicant. Power and regulated voltage of this mode can be adapted by effect levels. However, some applicants desire a sharper section during less pump clearance and subsequently a more distinct hemostasis. By adjustment of an initially higher voltage and power and reduction of these parameters triggered by the resistance determined at the instrument, the desired behavior can be realized. In doing so, such treatment options can be offered to the treating person, if he creates individual mode data for this that he can use subsequently for himself and, if desired, he can license to colleagues.

With the proposed device and the proposed method for programming of a device a possibility is created that widens the spectrum of usable instruments in that predefined modes are adapted to other instruments in the context of individual mode creation.

These and further advantages of embodiments of the invention as well as modifications and advanced embodiments are derived from the drawings, the description and the claims. The drawings show:

### BRIEF DESCRIPTION OF THE DRAWINGS

FIG. 1 shows an embodiment of the inventive apparatus for supply of an instrument in a functional block diagram illustration,

FIG. 2 shows the time-dependent progress of the generator voltage and the current received by the instrument in an exemplary application in form of a diagram,

FIG. 3 shows the time-dependent progress of the voltage and the current as according to FIG. 2, however, with a modification carried out by the user,

FIG. 4 shows the time-dependent progresses of the generator voltage for different tissue resistances; and

FIG. 5 shows the time-dependent progresses of the generator voltage for different tissue resistances, however, with a modification carried out by the user.

### DETAILED DESCRIPTION

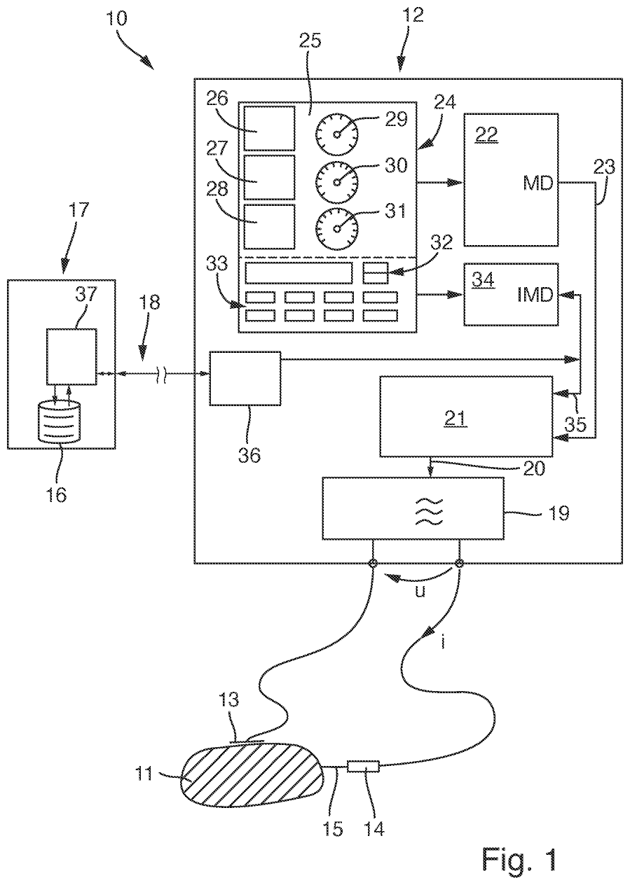

FIG. 1 shows a device 10 that serves for treatment of a human or animal patient 11 that is illustrated in FIG. 1 only symbolically in form of a cross-section of an arbitrary body part, on which an electrosurgical treatment is carried out. An apparatus 12 to which a neutral electrode 13 and an instrument 14 is connected is part of the device 10. The instrument 14 comprises at least one electrode 15 via which a treatment current is transferred on the patient 11 that is conducted out of the patient and back to the apparatus 12 by the neutral electrode 13. Thus, the instrument 14 is a monopolar instrument. Embodiments of the invention are, however, not limited to monopolar instruments, but can also be used in bipolar or multipolar instruments. Also the instrument 14 can be an instrument configured for surgical use, for the laparoscopic use or for the endoscopic use.

In addition, an external memory 16 is part of the device 10, i.e. a memory provided outside of the apparatus 12 that can be, for example, part of an external server 17. The server 17 and/or the memory 16 can be connected with the apparatus 12 temporarily or continuously via a data connection 18. The data connection 18 can be a wired electrical, an optical data connection or a radio connection or a combination thereof. Particularly the data connection can be realized via the internet.

The external server 17 can be realized in form of a separate hardware or as virtual machine within a hardware that comprises multiple servers and serves multiple customers. Particularly it can be provided that the external server 17 is connected or can be connected with multiple apparatus 12 that are located at different locations, particularly also in different hospitals or treatment centers. It is possible that all apparatus 12 of one manufacturer configured for this are connected or can be connected with the same server 17.

The apparatus 12 comprises a generator 19 for supply of the instrument 14 with radio frequency voltage U such that a respective radio frequency current i can flow via the patient 11. In order to provide the voltage U and/or the current i in the desired form, the generator 19 comprises a control input 20 that is connected with a control device 21. The control device 21 is configured to control the generator 19 according to mode data MD that are provided for this purpose in a respective memory 22. A memory section of a computer-supported apparatus control can serve as memory 22, wherein the apparatus control realizes all of the above and below-described functions, particularly also the functions of the control device 21.

The mode data predefine a number of physical parameters of the voltage U and/or the current i, wherein the control device 21 is configured to control the generator 19 according to this demand. The transmission of the mode data MD from the assigned memory 22 to the control device 21 is symbolized in FIG. 1 by an arrow 23.

A selection device 24 is part of the apparatus 12 and particularly part of its control, wherein the selection device 24 is configured to select a mode and for this comprises, for example, a control panel with real control elements or a touchscreen 25 with virtual control elements. Also each other suitable device for representation of symbols, such as a touchpad, a mobile phone or another input/output device is suitable as selection device 24. The selection device 24 can be part of the apparatus 12 or can be configured separately thereof and can then be connected therewith via a data connection.

The selection device 24 allows the selection of different modes by respective control elements 26, 27, 28. For example, the mode 26 can be a cutting mode, the mode 27 a precision cutting mode and the mode 28 a coagulation mode. For example, three monopolar cutting modes (autoCUT, highCUT, dryCUT), two endoscopic cutting modes (endoCUTQ, endoCUTI) and two bipolar cutting modes (autoCUT bipolar, highCUT bipolar) are distinguished in real apparatus of the applicant. For coagulation eleven different modes have been established in total (softCoag, swiftCoag, TwinCoag, preciseSect, sprayCoag, forced APC, pulsed APC, precise APC, soft Coag bipolar, forced Coag bipolar, Thermosil). A further selection switch 29, 30, 31 or a respective input panel can be assigned to each selectable mode 26, 27, 28 or its selection switch, via which the intensity of a mode can be adjusted. The intensity or effect strength is thereby typically the exclusive selectable and definable value for the respective selected mode 26, 27 or 28.

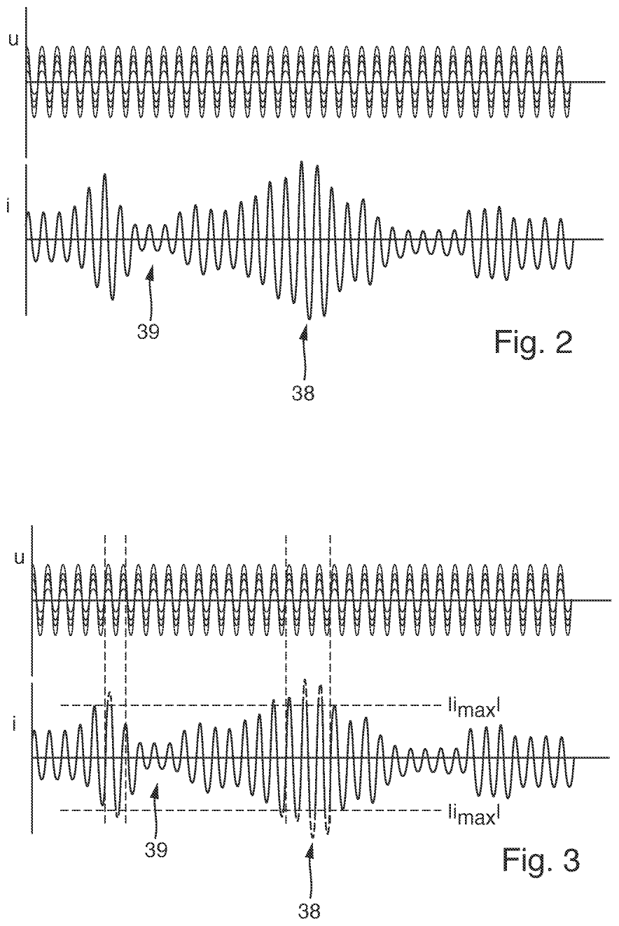

For this reference is made to FIG. 2. It illustrates the progress of the voltage U at the connection for the instrument 14. Only by way of the example a mode with non-modulated radio frequency voltage, e.g. a cutting mode, is illustrated for this purpose. The different adjustable effect strengths are assigned to different peak voltages of the radio frequency voltage that the generator 19 creates. With low effect strength the radio frequency voltage U has a small peak value. With high effect strength it has a high peak value. For example, the peak value can be selected via the input means 29.

In other modes, e.g. the precision cutting mode or the coagulation mode and also in general, the effect strength that can be selected at the respective input means 29, 30, 31 can also refer to other physical parameters. Examples are: internal resistance of the generator, maximum current, minimum current, maximum power, minimum power, transformed electrical work, crest factor, modulation type, pulse-pause-ratio or duty cycle with on/off modulation, rise durations and/or fall durations of a single radio frequency voltage impulse, repetition rates of pulses or pulse sequences, maximum switch-on time, duration of an increased power at the start of activation, amount of the activated power at the start of the activation and the like. However, in any case the manner how the input means effects the just mentioned physical parameters in order to influence the effect strength is set and cannot be varied with the previously discussed input means and selection switches.

The modes provided by the apparatus 12 in the context of the so far described adjustment possibilities are approved and certified by the manufacturer of the apparatus. A precise mode description exists for each of these modes 26, 27, 28 and for each additional generally provided mode that is provided in the memory of the apparatus 12 or can be downloaded from server 17. The treating person can thus quickly and reliably inform about the provided modes and its manner of application. The limitation of the operation types of the generator 19 to the mode data MD provided in the memory 22 thus serves for the purpose of patient safety. It however limits the application possibilities of the apparatus 12.

According to embodiments of the invention, it is provided that the treating person can use additional individual modes of the apparatus 12 that are not generally certified and released. For this the selection device 24 comprises an authentication device 32 via which the user can set up an own user account and can then always log in in his user account, e.g. by name and password. This user account is preferably primarily set up in server 17. In addition or as an alternative, it can also be provided in the apparatus 12 as original or copy. If the user has signed on at the apparatus 12, he gets direct access to physical parameters of the voltage U or the current i via additional control panels 33 or other input means, such as for example: maximum or minimum peak voltage, maximum or minimum peak current, maximum or minimum power, maximum or minimum work that has been carried out, maximum or minimum switch-on duration, cutting start parameters (current, voltage or other values during start of cutting), duty cycle, response time, decay time, pause durations, feedback control parameters, etc. For example, the treating person can select a preset and provided mode and vary it beyond the adjustment of the effect strength. On this basis the treating person creates individual mode data IMD that can be provided in an assigned memory 34. The control panels 33 and the software modules connected therewith thus form an individual mode generator.

The memory 34 can be a memory area of the apparatus control or also a separate memory. The individual mode data are transmitted to the control device 21 during activation, which is symbolized in FIG. 1 by arrow 35. In doing so; the treating person can create a modified or completely individual mode and use it for his treatment purposes in the context of the medical freedom of treatment.

In addition, the apparatus 12 can comprise a communication module 36 that is connected or can be connected with the communication block 37 of the server 17 via the data connection 18. The communication module 36 is thereby configured to transmit the individual mode data IMD created by the authenticated treating person to the server 17 and via the communication block 37 to the memory 16.

The server 17, however, serves at least preferably not only for the passive storage of the individual mode data IMD created by the authenticated user. Rather the communication block 37 can obtain support during the creation of individual mode data IMD by means of suitable support programs. For example, means for revision of the individual mode data IMD can be provided that indicate a thermal or electrical overload of the instrument 14, of the generator 19 or also an expected injury of the patient 11. Such means can be simple comparators that, for example, indicate an access of power thresholds on the patient or of maximum voltages or maximum currents. It can also be an artificial intelligence on the basis of algorithms or also an artificial intelligence that is able to learn, that outputs warnings based on multiple individual mode data created by different treating persons, if it is significantly deviated from otherwise created individual mode data IMD. The communication block 37 can also comprise an interface for a human treating person, who is, for example, available as medical and technical skilled person as contact person and revises the individual mode data IMD.

For explanation of the operation on basis of individual mode data IMD reference is first made to FIG. 2. Thereby it is assumed that the user has first selected a common certified mode, e.g. the cutting mode 26 and thereby the highest effect strength, i.e. the highest voltage peak value. During the work on the patient the current progress illustrated in FIG. 2 may result. This current progress is minimum, if the current flows through tissue with high resistance and is maximum, if a current flows through tissue with low tissue resistance. For clarification of embodiments of the invention it is now assumed that the resulting peak currents, e.g. at 38 in FIG. 2, are undesired. The treating person could avoid such peak currents in that he adjusts a lower effect strength. However, this would result in too low currents in tissues with high resistance, as for example at 39. In embodiments of the inventive apparatus the treating person has now the possibility for the creation of an individual mode, as illustrated in FIG. 3. For this he defines a maximum absolute value for the peak current |iₘₐₓ|, for example. In doing so, he has created an individual mode in which with maximum effect strength the current, however, does not exceed a maximum absolute value |iₘₐₓ|.

The treating person can store this individual mode in the memory 34 and can make it accessible for later repetition. For this he has to authenticate on the apparatus 12. This individual mode is only provided for the treating person in the context of its medical freedom of treatment that is authenticated for the use of this individual mode. Such a treating person is either the creator of the individual mode or a person that has obtained the authority for use.

The treating person can, however, provide the individual mode data IMD in the server 17 for download also for colleagues. His individual mode data IMD get an identifier for this purpose, e.g. a name, that the authenticated treating person inputs via the input device 25 and uploads it on server 17. For this the authenticated treating person can also define the conditions of reuse, e.g. license conditions, or select them from a predefined list and link them with his individual mode.

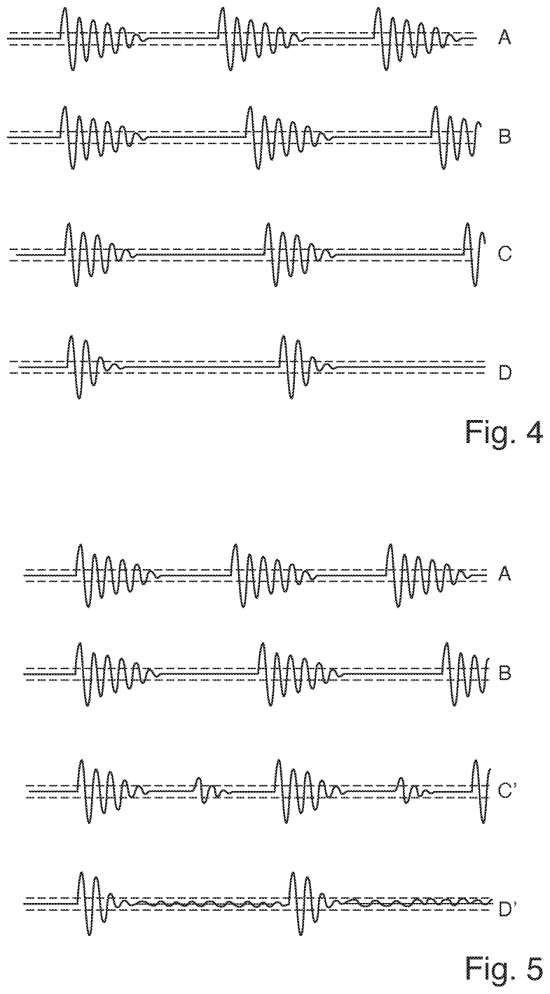

The creation of individual mode data IMD is not limited to the limitation of voltage or current or other electrical parameters. Also qualitative modifications can be carried out on modes or individual modes can be created. For this reference is made to FIGS. 4 and 5. FIG. 4 illustrates the precision cut mode 27 in which the voltage U adapts to determined tissue resistances. In case of a very high tissue resistance, the voltage progress A results. With decreasing tissue resistance the voltage progress changes toward greater distances and shorter impulses until a voltage progress D is reached in case of a very low tissue resistance.

If the treating person recognizes, for example, that he has an undesired behavior of the instrument 14 (e.g. too high cutting resistance) in case of low and very low tissue resistance (C and D), he can modify the selected cutting mode for authentication. For example, he can insert attenuated pulses in the pulse pauses in case of low tissue resistance, as illustrated in FIG. 5 in voltage progress C′. In case of a very low tissue resistance, he can for example, apply a low radio frequency voltage in the pulse pauses, as only by way of example illustrated in the voltage progress D′. Also other parameters of the voltage progress and/or current progress or other electrical parameters can be varied.

Also here it applies again that an individual mode created in this manner can be made accessible for other treating persons, in that this mode is loaded as noncertified individual mode on server 17 and is provided there for reuse after licensing.

Embodiments of the inventive device for surgical treatment of patients comprises an apparatus 12 having an electrical generator 19 at which different modes 26, 27, 28 can be selected. In addition to selection of provided modes 26, 27, 28 and for adjustment of their effect strength, the authenticated treating person can generate at least one individual mode and associated individual mode data IMD for himself and if applicable also selected colleagues as reusers in the apparatus 12 based on which the generator 19 operates beyond certified modes. In doing so, treatment possibilities are offered for the treating person that would otherwise be refused to him. The generation of the individual modes can be carried out without restrictions in one embodiment, such that the authenticated treating person can carry out all adjustments completely free within the physical limits of the apparatus. It is also possible that the apparatus control limits the adjustment possibilities in order to limit or exclude hazards for patients and personnel. It is further possible that the apparatus control presets different limitations of the adjustment possibilities based on different authentications.

1. A device for surgical treatment of animal or human patients, the device comprising:
an apparatus for supply of at least one instrument, the apparatus comprising:
a generator configured to create a voltage for supply of the instrument with electric current; and
a control device that is configured to control the generator according to selectable modes stored in a mode memory and provided for selection,
wherein mode data are assigned to each selectable mode that define at least one of a number of physical parameters of the current and the voltage and
wherein the control device is configured to control the generator according to the mode data of a selectable mode,
a selection device that is configured for manual selection of a mode and that is in addition configured for at least some of the selectable modes for adjustment of a desired effect strength;
an external memory arranged outside of the apparatus in which individual mode data can be stored that are assigned to individual modes that are individually assigned to selected treating persons; and
an authentication device configured to individually identify treating persons and to allow the transmission of individual mode data of the authenticated treating person on the apparatus and/or the access to individual mode data stored in an individual mode memory of the apparatus for control of the generator or to create such individual mode data and that is further configured to block or delete individual mode data, if the authentication terminates.

2. The device according to claim 1, wherein the control device is configured to influence at least one of the physical parameters (voltage u, current I, power P, work W) of the selected mode based on the adjusted effect strength.

3. The device according to claim 1, wherein the external memory is part of a server that is connected or can be connected with the apparatus via a data connection.

4. The device according to claim 1, wherein the authentication device is configured to provide a check-out option for determination of the authentication.

5. The device according to claim 1, wherein a mode generator is provided for generation of selectable modes that detects user inputs of an authenticated treating person with regard to physical parameters of the voltage and/or the current and is configured to generate assigned individual mode data.

6. The device according to claim 5, wherein the individual mode data refer to such physical parameters of the current or the voltage that are beyond the available adjustment possibilities in the context of selection of the mode and effect strengths.

7. The device according to claim 5, wherein the individual mode data can be transferred together with an identifier identifying the authenticated treating person to the external memory.

8. The device according to claim 7, wherein the individual mode data assigned to an authenticated treating person stored in the external memory can be made accessible for another authenticated treating person for a download from the external memory into the individual mode memory.

9. A method for operating a treatment device, the method comprising:
a generator of an apparatus for supply of at least one instrument generating a voltage; and
the generator supplying a current to the instrument,
wherein the generator is controlled according to selectable modes stored in an individual mode memory and provided for selection,
wherein mode data that define a number of physical parameters of the voltage and/or the current that cannot be modified by the user and effect strengths adjustable by the user are part of each selectable mode, and
wherein a treating person is identified individually by means of an authentication device, whereby the transmission of individual mode data of the authenticated treating person to the apparatus and/or the access to individual mode data stored in the individual mode memory of the apparatus for control of the generator or the creation of such individual mode data is allowed and wherein further the individual mode data are blocked or deleted, if the authentication terminates.

10. The method according to claim 9, wherein the individual mode data are stored in an external memory arranged outside the apparatus that are part of individual modes that are assigned individually to selected treating persons.

11. The method according to claim 9, wherein the individual mode data are stored on a server that is connected or can be connected with the apparatus via a data connection.

12. The method according to claim 9, wherein the authentication is terminated by means of a check-out option.

13. The method according to claim 9, wherein an individual mode is created by means of a mode generator that detects user inputs of an authenticated treating person with reference to physical parameters of the voltage and/or the current and generates assigned individual mode data.

14. The method according to claim 9, wherein such physical parameters of the current or the voltage are defined for generation of the individual mode data that are beyond the available adjustment possibilities in the context of the selection of the modes and effect strengths.

15. The method according to claim 9, wherein the individual mode data are transmitted together with an identifier identifying the authenticated treating person to the external memory and that the individual mode data assigned to an authenticated treating person stored in the external memory are released for download for another authenticated treating person into the individual mode memory.
